(19) United States
(12) Patent Application Publication
(10) Pub. No.: US 2016/0175348 A1
(43) Pub. Date: Jun. 23, 2016

(54) ENHANCED NITRIC OXIDE DELIVERY AND USES THEREOF

(71) Applicant: ALBERT EINSTEIN COLLEGE OF MEDICINE, INC., Bronx, NY (US)

(72) Inventors: Adam Friedman, New York, NY (US); Joel Friedman, South Orange, NJ (US); Joshua Nosanchuk, Upper Saddle River, NJ (US); Parimala Nacharaju, Staten Island, NY (US); Karin Blecher, Little Silver, NJ (US); Chaim Tuckman-Vernon, West Orange, NJ (US)

(21) Appl. No.: 15/055,351

(22) Filed: Feb. 26, 2016

Related U.S. Application Data
(63) Continuation of application No. 14/800,415, filed on Jul. 15, 2015, now abandoned.
Application No. 14/800,415 is a continuation of application No. 14/575,335, filed on Dec. 18, 2014, now abandoned.
Application No. 14/575,335 is a continuation of application No. 14/250,099, filed on Apr. 10, 2014, now abandoned.
Application No. 14/250,099 is a continuation of application No. 13/533,025, filed on Jun. 26, 2012, now abandoned.
(60) Provisional application No. 61/501,291, filed on Jun. 27, 2011.

(51) Int. Cl. A61K 33/00; A61K 9/48; A61K 38/06

(52) U.S. Cl. CPC A61K 33/00; A61K 38/063; A61K 9/48

(57) ABSTRACT

Methods and compositions are disclosed that enhance delivery of nitric oxide (NO) by combining nitric oxide releasing nanoparticles (NO-np) with exogenous glutathione (GSH), as well as therapeutic uses of the methods and compositions.

### CROSS-REFERENCE TO RELATED APPLICATION

This application claims the benefit of U.S. Provisional Patent Application No. 61/501,291, filed on Jun. 27, 2011, the contents of which are herein incorporated by reference.

### FIELD OF THE INVENTION

The present invention relates generally to methods and compositions to enhance delivery of nitric oxide (NO) by combining nitric oxide releasing nanoparticles (NO-np) with exogenous glutathione (GSH), and therapeutic uses of the methods and compositions.

### BACKGROUND OF THE INVENTION

Throughout this application various publications are referred to in brackets. Full citations for these references may be found at the end of the specification. The disclosures of these publications are hereby incorporated by reference in their entirety into the subject application to more fully describe the art to which the subject invention pertains.

Nitric oxide (NO) is a vital component of mammalian host defense, produced in and by cells comprising the innate immune system, most importantly macrophages. NO is unique as an antimicrobial agent as it can inhibit or kill a broad range of microorganisms [1; 2]. NO can interact with and alter protein thiols and metal centers [3; 4], blocking essential microbial physiological processes, including respiration and DNA replication [5; 6; 7]. Peroxynitrite (ONOO) [6; 8; 9], which is formed by oxidation of NO, nitrogen dioxide (NO₂), dinitrogen tridioxide (N₂O₃), and nitroxyl ions induce oxidation of key pathogen machinery. Furthermore, these species can initiate continuous lipid peroxidation reactions, adding to NO's antimicrobial power.

In bacteria subjected to this n trosative stress, S-nitrosoglutathione (GSNO) is formed by reaction of NO with intracellular glutathione [10; 11; 12]. GSNO is fundamentally a NO-donor that can spontaneously transfer NO to other thiols. GSNO, a S-nitrosothiol, is different from other NO donors because it contributes to the transnitrosation and sulfhydryl formation of enzymatic proteins; a process that results in the reversible blockade of thiol groups on enzymes [13]. Most pharmacological actions of nitrosothiols are a consequence of this nitrosation of cellular proteins that are essential to many physiologic processes. In fact, GSNO is the one of the most effective trans-nitrosating agents under physiologic conditions [2]. To control the level of S-nitrosylated proteins and protect cellular machinery, organisms use GSNO reductases and nitroreductases [14]. Pathogens also rely on the regenerated glutathione (GSH) for protection against oxidative damage.

Staphylococcus aureus is the most common drug resistant Gram positive pathogen responsible for a large number of human infections. The rising incidence of hospital-, and more recently, community-acquired methicillin resistant S. aureus (MRSA) infections has led to a medical crisis of epidemic proportions, highlighting the need for new and innovative therapies [15; 16; 17; 18; 19]. This emergency is further propagated by the evolving resistance of Gram negative pathogens such as Escherichia coli, Klebsiella pneumoniae and Pseudomonas aeruginosa. A nanoparticulate platform capable of controlled and sustained release of NO (nitric oxide releasing nanoparticles (NO-np)) significantly and effectively kills both Gram positive and negative organisms in vitro [20; 21] and accelerates clinical recovery in vivo in murine wound and abscess infection models [20; 21; 22]. Interestingly, in vivo efficacy of the NO-np outmatched in vitro data generated, likely due to the diverse and multifaceted impact of NO in a living system. One such interaction is NO combining with host and pathogen GSH to form GSNO, which provides for both a more stable form of NO and, more importantly, a potent nitrosating agent. In fact, NO itself can not act as a nitrosating agent, rather it relies on nitrosating agents such as GSNO to transfer the nitrosonium group (NO+) to a nucleophilic receptor such as an amine or thiol [23]. It is this transfer that results in pathogen DNA or enzymatic damage, ultimately impeding microbial survival.

The present invention addresses the need for methods and compositions for improved delivery of nitric oxide for a variety of therapeutic applications, including for example treatment of pathogens.

### SUMMARY OF THE INVENTION

The present invention provides methods and compositions for enhancing the efficacy of nitric oxide (NO) released from NO releasing nanoparticles (NO-np) comprising combining the NO-np with exogenous glutathione (GSH) so as to enhance the efficacy of NO that is released. The methods and compositions can be use in a variety of therapeutic applications such as, for example, treating microbial infections, cutaneous inflammatory disorders such as but not limited to psoriasis and eczema, burns, erectile dysfunction, cardiovascular disorders, pulmonary disorders, peripheral vascular disease, scleroderma, or sickle cell anemia, and/or promoting wound healing, hair growth, angiogenesis, or vasodilation.

### BRIEF DESCRIPTION OF THE DRAWINGS

FIG. 1a-1b. GSNO formation from NO-np and GSH. (a) RPHPLC analysis of the NO-np and GSH reaction mixture. NO-np (5 mg/ml) were incubated in 20 mM GSH and 0.5 mM DTPA in phosphate buffered saline (PBS) for 30 minutes at room temperature. The supernatant was analyzed by RPHPLC as described in the methods section (solid line). Peaks 1 through 4 are identified as GSH, nitrite, GSSG, and GSNO, respectively. The unidentified small peaks appear to be various oxidation products of GSH/GSNO. The dotted line in the chromatogram corresponds to 1 mM GSNO stock. (b) Time course of GSNO formation. NO-np (5 mg/ml) were incubated in GSH (20 mM) in DTPA (0.5 mM) in PBS at room temperature. Aliquots were analyzed at time intervals on RPHPLC and the GSNO concentration was calculated from GSNO peak area. The observed GSNO concentration (solid line) and the calculated actual GSNO concentration (dotted line), derived by adjusting the GSNO decay as described in the methods section, are plotted as a function of time.

FIG. 2a-2b. MRSA is susceptible to NO-np. (a) Susceptibility of MRSA isolates (n=5) to NO (NO-np 5 mg/ml), GSNO (NO-np 5 mg/ml+10 mM GSH) empty nanoparticles (np) was investigated by real-time Bioscreen analysis and (b) percent survival determined by colony forming unit assays following 24 hours incubation. The data shown are an average of the results from the bacterial isolates tested in triplicates and error bars represent standard error from mean. Each point (fig a) represents the average of four measurements of four identical wells and error bars denote standard error from mean. Experiments were repeated in triplicate and performed at least twice on separate days. Asterisks denote p value significance (*P<0.0001) calculated by unpaired two-tailed t test analysis.

FIG. 3a-3b. E. coli is susceptible to NO-np and GSNO. (a) Susceptibility of E. coli isolates (n=3) to NO (NO-np 5 mg/ml), GSNO (NO-np 5 mg/ml+10 mM GSH) or np was investigated by real-time Bioscreen analysis and (b) CFU determinations at 24 hours of growth. Experiments were repeated in triplicate and performed at least twice on separate days. Asterisks denote p value significance (*P value=0.003; **P value=0.0001) calculated by unpaired two-tailed t test analysis.

FIG. 4a-4b. K. pneumoniae is susceptible to NO-np and GSNO. (a) Susceptibility of K. pneumoniae isolates (n=3) to NO (NO-np 5 mg/ml), GSNO (NO-np 5 mg/ml+10 mM GSH) or np was investigated by real-time Bioscreen analysis and (b) CFU determinations at 24 hours of growth. Experiments were repeated in triplicate and performed at least twice on separate days, Asterisks denote p value significance (*P value=0.0001; **P value=0.0002) calculated by unpaired two-tailed t test analysis.

FIG. 5a-5b. P. aeruginosa is susceptible to NO-np and GSNO (a) Susceptibility of P. aeruginosa isolates (n=3) to NO (NO-np 5 mg/ml), GSNO (NO-np 5 mg/ml+10 mM GSH) or np was investigated by real-time Bioscreen analysis and (b) CFU determinations at 24 hours of growth. Experiments were repeated in triplicate and performed at least twice on separate days. Asterisks denote p value significance (*P value=0.0001; **P value<0.0001) calculated by unpaired two-tailed t test analysis.

FIG. 6a-6b, Bioscreen (a) and CFU (b) assays demonstrate that the NO-np+GSH combined treatment are significantly superior in inhibiting growth as well as killing clinical isolate of Pseudomonas aeruginosa as compared to controls and NO-np alone in vitro.

FIG. 7a-7b. NO-np (10 mg/ml)+GSH (10 mM) is more effective than controls or NO-np alone in a Pseudomonal excisional wound mouse model. (a) Visual changes in would over time. (b) Percent change in wound area (upper) and CFU assay (lower).

### DETAILED DESCRIPTION OF THE INVENTION

The invention provides a method for enhancing the efficacy of nitric oxide (NO) released from NO releasing nanoparticles (NO-np) comprising combining the NO-np with exogenous glutathione (GSH) so as to enhance the efficacy of NO that is released.

The invention also provides a composition for delivering nitric oxide (NO) comprising NO releasing nanoparticles (NO-np) and glutathione (GSH).

Preferably, S-nitrosoglutathione (GSNO) is formed by reaction of NO with GSH.

GSH can be dissolved in a carrier mixed with NO-np, and/or GSH can be encapsulated in nanoparticles (GSH-np) and mixed with NO-np.

Methods of producing NO releasing nanoparticles (NO-np) have been described in, for example, U.S. Patent Application Publication No. 2009/0297634 and PCT International Publication No. WO 2010/123547, the contents of which are herein incorporated by reference.

For example, NO-np can comprise nitric oxide encapsulated in a matrix of chitosan, polyethylene glycol (PEG) and/or polyvinyl alcohol (PVA), and tetra-methoxy-ortho-silicate (TMOS) or tetra-ethoxy-ortho-silicate (TEOS). Another composition for releasing nitric oxide (NO) comprises nitric oxide encapsulated in a matrix of trehalose, and non-reducing sugar or starch. The composition can further comprises nitrite, reducing sugar, and/or chitosan. Another composition for releasing nitric oxide (NO) comprises nitrite; reducing sugar; chitosan; polyethylene glycol (PEG) and/or polyvinyl alcohol (PVA); tetra-methoxy-ortho-silicate (TMOS) or tetra-ethoxy-ortho-silicate (TEOS); and nitric oxide encapsulated in a matrix of chitosan, PEG and TMOS. Another composition for releasing nitric oxide (NO) comprises nitrite; reducing sugar; chitosan; trehalose; a non-reducing sugar or starch; and nitric oxide encapsulated in a matrix of trehalose and the non-reducing sugar or starch. Another composition comprises nitrite, reducing sugar, chitosan, polyethylene glycol (PEG) and tetra-methoxy-ortho-silicate (TMOS) or tetra-ethoxy-ortho-silicate (TEOS), and a composition comprising nitrite, reducing sugar, chitosan, trehalose, and non-reducing sugar or starch. Nitric oxide is released when the composition is exposed to an aqueous environment.

The NO-np can comprise a silane in addition to TMOS or TEOS. The additional silane can be chosen, for example, to either alter the internal environment of the resulting particles with respect to properties such as hydrophobicity and polarity or to introduce reactive groups (e.g. amino, carboxyl, sulfhydryl) that allow the covalent attachment of additional molecules to the particles. The additional silane can be, for example, a hydrophobic silane, such as, for example, trimethoxyalkyl isopropyl silane, trimethoxyalkyl butyl silane or trimethoxyalkyl fluoropropyl silane.

One method for preparing NO-np comprises, for example: (a) admixing nitrite, reducing sugar, chitosan, polyethylene glycol (PEG) and/or polyvinyl alcohol (PVA), and tetra-methoxy-ortho-silicate (TMOS) or tetra-ethoxy-ortho-silicate (TEOS); (b) drying the mixture of step (a) to produce a gel; and (c) heating the gel until the gel is reduced to a powdery solid. The nitrite is reduced to nitric oxide by the reducing sugar, and nitric oxide is encapsulated in the powdery solid. The encapsulated nitric oxide is released when the composition is exposed to an aqueous environment. The solid of step (c) can be ground to produce particles of a desired size. Preferably, the gel is heated in step (c) to a temperature of 55-70° C., more preferably to about 60° C. Preferably, the gel is heated in step (c) for 24-28 hours. Another method for preparing a composition for releasing nitric oxide (NO) comprises: (a) admixing nitrite, reducing sugar, chitosan, trehalose, and non-reducing sugar or starch; (b) drying the mixture of step (a) to produce a film; and (c) heating the film to form a glassy film. The nitrite is reduced to nitric oxide by the reducing sugar, and nitric oxide is encapsulated in the glassy film. The encapsulated nitric oxide is released when the composition is exposed to an aqueous environment. Preferably, the film is heated in step (c) to a temperature of 55-70° C., more preferably to about 65° C. Preferably, the film is heated in step (c) for about 45 minutes. Preferably, the nitrite is a monovalent or divalent cation salt of nitrite, including for example, one or more of sodium nitrite, calcium nitrite, potassium nitrite, and magnesium nitrite. Preferably, the concentration of nitrite in the composition is 20 nM to about 1 M. The gel can also be lyophilized to produce a particulate material. Alternatively, the mixture may be spray dried to produce a particulate material.

As used herein, a “reducing sugar” is a sugar that has a reactive aldehyde or ketone group. The reducing sugar is used to reduce nitrite to nitric oxide. All simple sugars are reducing sugars. Sucrose, a common sugar, is not a reducing sugar. Examples of reducing sugars include one or more of glucose, tagatose, galactose, ribose, fructose, lactose, arabinose, maltose, and maltotriose. Preferably, the concentration of reducing sugar in the composition is 20 mg-100 mg of reducing sugar/ml of composition.

Preferably, the chitosan is at least 50% deacetylated. More preferably, the chitosan is at least 80% deacetylated. Most preferably, the chitosan is at least 85% deacetylated. Preferably, the concentration of chitosan in the composition is 0.05 g-1 g chitosan/100 ml of composition (dry weight).

Preferably, the concentration of TMOS or TEOS in the composition is 0.5 ml-5 ml of TMOS or TEOS/24 ml of composition (dry weight).

Preferably, the polyethylene glycol (PEG) has a molecular weight of 200 to 20,000 Daltons, more preferably 200-10,000 Daltons, and most preferably 200-5,000 Daltons. In different embodiments, the PEG can have a molecular weight of, for example, 200-400 Daltons or 3,000-5,000 Daltons. PEGs of various molecular weights, conjugated to various groups, can be obtained commercially (see, for example, Nektar Therapeutics, Huntsville, Ala.). Preferably, the concentration of polyethylene glycol (PEG) in the composition is 1-5 ml of PEG/24 ml of composition (dry weight).

The nanoparticles can be formed in sizes having a diameter in dry form, for example, of 10 nm to 1,000 μm, preferably 10 nm to 100 μm, or 10 nm to 1 μm, or 10 nm to 500 nm, or 10 nm to 100 nm.

Preferably, the NO-np are nontoxic, nonimmunogenic and biodegradable.

The NO-np and GSH can be delivered to a subject by a variety of routes of delivery, including but not limited to percutaneous, inhalation, oral, intraperitoneal, intravenous, local injection, and aerosol administration. The compositions can be incorporated, for example, in a cream, lotion, ointment, solution, foam, oil, transdermal patch, implantable biomedical device, facial patch or facial scrub.

The invention also provides methods of treating an infection in a subject, such as a microbial infection, comprising administering to the subject NO-np and GSH effective to treat the infection. The term “infection” is used to include infections that produce an infectious disease. The infection diseases include communicable diseases and contagious diseases. As used herein, the term “treat” an infection means to eliminate the infection, to reduce the size of the infection, to prevent the infection from spreading in the subject, or to reduce the further spread of the infection in the subject.

The infection can be, for example, a bacterial, viral, fungal or parasitic infection. The bacterial infection can be caused, for example, by a bacterium selected from the group consisting of S. aureus, B. circulans, B. cereus, Escherichia coli, P. vulgaris, P. acnes, S. pyognenes, S. enterica, V. anguillarum, Kiebsiella pneumoniae, P. piscicida, Pseudomonas aeruginosa, A. tumefaciens, C. micgiganence, A. mali, E. chrysanthemi, E. campestris, C. diplodiella, P. piricola, M. tuberculosis, M. ulcerans and methicillin resistant Staphylococcus aureus (MRSA). The fungal infection can be caused, for example, by a fungus selected from the group consisting of T. equinum, C. Albicans, F. oxysporum, R. solani, B. cinerea, and A. flavus. The viral infection can be caused, for example, by a virus selected from the group consisting of M. contagiosum, Rota, Papilloma, Parvo, and Varicella. The parasite infection can be caused, for example, by a parasite of the genus Plasmodium, Leishmania, Schistosoma, Austrobilharzia, Heterobilharzia, Ornithobilharzia or Cryptosporidium, for example P. falciparum.

The invention also provides methods of promoting angiogenesis, vasodilation, wound healing, or hair growth in a subject comprising administering to the subject NO-np and GSH effective to promote angiogenesis, vasodilation, wound healing, or hair growth.

The invention also provides methods of administering nitric oxide (NO) to a subject comprising administering to the subject a combination of NO releasing nanoparticles (NO-np) and exogenous glutathione according to any of the methods disclosed herein. The invention further provides methods of treating a disorder in a subject comprising administering to the subject NO-np and GSH effective to treat the disorder. The subject can have, or the disorder can be, e.g., inflammatory skin disease such as but not limited to psoriasis and eczema, peripheral vascular disease, erectile dysfunction, scleroderma, a burn, sickle cell anemia, a cardiovascular disorder, a pulmonary disorder, a microbial infection, or a wound. The term “treat” a disorder means to reduce or eliminate a sign or symptom of the disorder, to stabilize the disorder, or to reduce further progression of the disorder.

NO-np and GSH can be applied directly to an affected area when treating, for example, a burn, a wound, hair loss or erectile dysfunction.

The invention further provides an antimicrobial agent, a wound healing acceicrant, a pro-erectile agent, an anti-hypertensive agent, a pro-resuscitive agent, a blood storage stabilizer, an anti-vasospastnic agent, a chemotherapeutic agent, an immunomodulatory agent, or an anti-aging agent comprising any of the compositions disclosed herein that contain NO-np and GSH.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Materials and Methods

NO Particle (NO-np) Synthesis:

The generation of NO-np has been previously reported [24; 25]. Briefly a hydrogel/glass composite was synthesized using a mixture of tetramethylorthosilicate (TMOS), polyethylene glycol (PEG), glucose, chitosan, and sodium nitrite in a 0.5 M sodium phosphate buffer (pH 7). The nitrite was reduced to NO within the matrix because of the glass properties of the composite effecting redox reactions initiated with thermally generated electrons from glucose. After redox reaction, the ingredients were combined and dried using a lyophilizer, resulting in a fine powder comprising nanoparticles containing NO. Once exposed to an aqueous environment, the hydrogel properties of the composite allow for an opening of the water channels inside the particles, facilitating the release of the trapped NO over extended time periods.

GSNO Formation Reaction:

NO-np were suspended (5 mg/ml) in 10 or 20 mM GSH and 0.5 mM DTPA in PBS. The reaction mixture was incubated at room temperature while mixing. At time intervals of 5, 30, 60, 120, 240, and 1440 minutes, 100 μl aliquots of the supernatant were removed from the reaction mixture and split into two portions. One was diluted twenty times and analyzed immediately on RPHPLC. The other was left at room temperature until the next time point to monitor the decay of GSNO and was then analyzed as described.

RPHPLC Analysis of the GSNO Formation Reaction:

The reaction products were analyzed by RPHPLC using a Vydac Protein and Peptide C₁₈ column (250 mm×10 mm) in an isocratic 10 mM K₂HPO₄/10 mM Tetrabutylammonium Hydrogen Sulfate in 5% Acetonitrile running buffer at a 1 ml/min flow rate and were detected by UV absorbance at 210 nm or 335 nm as indicated.

The amount of GSNO formed was calculated from the GSNO peak area using a known GSNO sample (Sigma, St Louis, Mo.) as a standard. GSNO decays rapidly to form the oxidized product GSSG in solutions at room temperature. It was therefore necessary to take the decay of GSNO into account when calculating the actual amount of GSNO formed in the reaction mixture over time. Therefore, in order to calculate the actual amount of GSNO formed at each time interval, the amount of GSNO decayed over the previous time period needed to be determined The amount of decay over the previous time period was calculated from the difference of GSNO concentrations obtained for the aliquot that was analyzed at the previous time point immediately and the one analyzed after leaving it for decay until the next time point (as described above). This difference was added to the observed GSNO at the next time point. The following general formula was used to calculate the actual concentration of GNSO: [GNSO]_{actual@t}=[GSNO]_{obs@t}+([GSNO]_{actual@t-1}−[GSNO]_{decay@t-1}).

Methicillin Resistant Staphylococcus Aureus (MRSA), E. Coli, K. Pneumoniae, and P. Aeruginosa, Clinical Isolates

All clinical isolates used were collected from Montefiore Medical Center, Bronx, N.Y. All samples were obtained with written consent of all patients according to the practices and standards of the institutional review boards at the Albert Einstein College of Medicine and Montefiore Medical Center. A total of 17 clinical isolates were studied including 5 MRSA (6524, 8166, 1115, 0570, 6205), 3 E. coli (8418, 5535, 7540), 3 K. penumoniae (0441, 8963, 4160), and 3 P. aeruginosa (1911, 0234, 1620). All strains were stored in Tryptic Soy Broth (TSB, MP Biomedicals, LLC, Solon, Ohio) containing 40% glycerol at −80° C. until use, and then grown in TSB broth overnight at 37° C. with rotary shaking at 150 r.p.m.

Susceptibility of MRSA, E. Coli, K. Kneumoniae, and P. Aeruginosa to NO-np and Combination NO-np and Glutathione (NO-np/GSH):

To determine the impact of the NO-np and combination NO-np and GSH (NO-np/GSH) on the various clinical isolates, TSB was inoculated with a one fresh colony of bacteria grown on tryptic soy agar (TSA) plates and suspended in 1 ml of medium. A bacterial suspension of 1 μL was transferred to a 100-well honeycomb plate with 199 μL of TSB per well containing 5 mg/ml NO-np or np, 5 mg/ml NO-np or np and 10 mM GSH, or 10 mM GSH alone. Prior to plating, NO-np, np, and combinations with GSH were sonicated for 1 minute on ice with a Fisher sonic Dismembrator (model 200, Fisher Scientific, Pittsburgh, Pa.). Controls included wells containing bacteria with TSB alone. The background OD of nanoparticles was accounted for by plating wells containing TSB and NO-np or np alone. Bacteria and nanoparticles were incubated for 24 hours at 37° C. and growth was assessed at an optical density (OD) of 600 nm every 30 minutes using a micropalate reader (Bioscreen C, Growth Curves USA, Piscataway, N.J.).

Colony Forming Unit (CFU) Assay:

After incubation with NO-np, 10 μL of suspension containing bacteria was aspirated from each experimental group and transferred to an eppendorf tube with 990 ml of phosphate-buffered saline (PBS) and vortexed gently. The suspensions were serially diluted in PBS and aliquots were plated on TSA plates. The percentage of CFU survival was determined by comparing survival of NO-treated bacterial cells relative to the survival of untreated bacteria. Minimum inhibitory concentration required to inhibit the growth of 90% of organisms (MIC₉₀) was determined using CFU assays as previously described [21].

Statistical Analysis:

All data were subjected to statistical analysis using GraphPad Prism 5.0 (GraphPad Software, La Jolla, Calif.). P-values were calculated by analysis of variance and were adjusted by use of the Bonferroni correction. P-values of <0.05 were considered significant.

### Results

GSNO is Generated from NO-np and GSH:

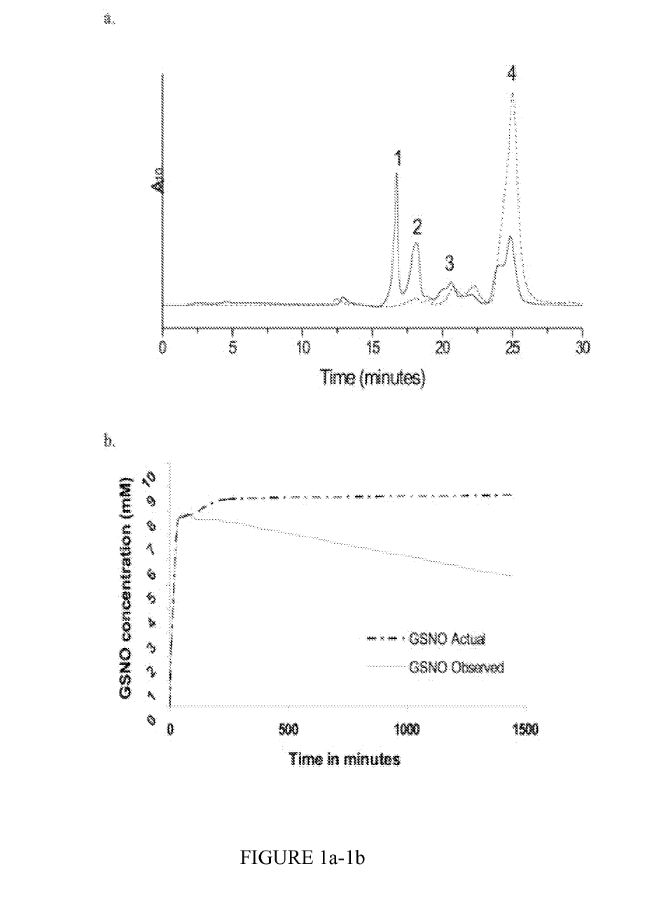

Based on the known nitrite content of the NO-np, 5 mg/ml suspension of NO-np can release a maximum of 15 mM NO over their entire course of activity. Two concentrations of GSH, 10 mM or 20 mM, were used to react with NO-np at 5 mg/ml (FIG. 1a). Components of the reaction mixture in the chromatogram (GSH, nitrite, GSSG, and GSNO) were identified by analyzing each of these components separately at known concentrations. GSSG is the oxidized product (dimer) of GSH. The curve corresponding to purified GSNO peak shown in the FIG. 1a was obtained by analyzing a purified GSNO sample.

The time course of formation of GSNO from a mixture of NOnp (5 mg/ml) and 20 mM GSH was demonstrated (FIG. 1b). Approximately 7.9 mM GSNO was formed in the first hour of the reaction. This concentration reduced to 5.33 mM over a period of 24 h due to the oxidation of GSNO to GSSG. However, by accounting for the amount of GSNO oxidation, an increase in the concentration of GSNO to 8.67 mM was observed over this period, indicating the sustained release of NO from particles and progress of the reaction. At least a 20 fold lower amount of GSNO (˜300 μM) formed when 10 mM GSH was used with 5 mg/ml NO-np.

NO-np and NO-np with GSH Inhibit MRSA Growth/Survival:

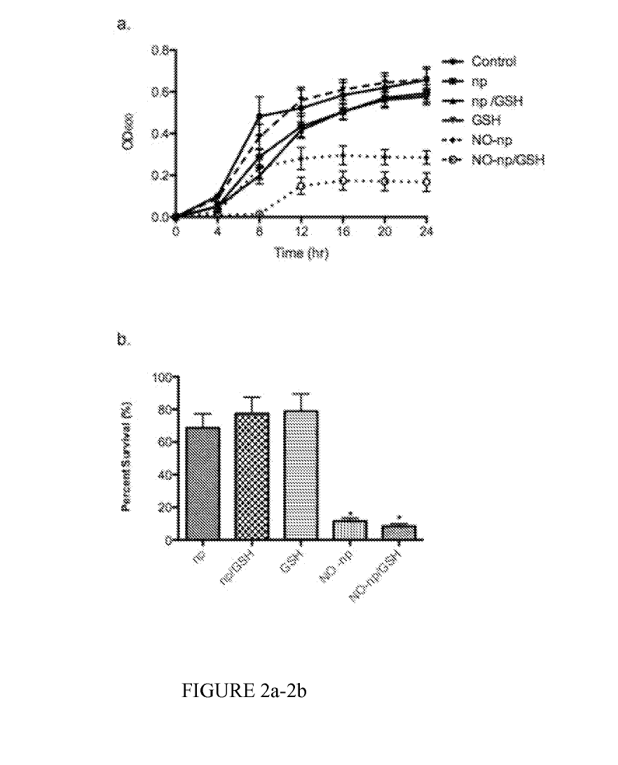

The effect of NO-np and NO-np/GSH on MRSA growth was determined in real-time for 24 h by Bioscreen C analysis (FIG. 2a). All isolates were challenged with a NO-np concentration of 5 mg/ml, with or without 10 mM GSH, as this NO-np concentration consistently demonstrated efficacy in both past in vitro and in vivo studies without any evidence of host cellular or tissue damage [21; 22; 25]. At the 5 mg/ml NO-np concentration, both NO-np alone and NO-np/GSH significantly limited bacterial growth after 24 h co-incubation for all isolates, which correlated with CFU assays (FIG. 2b). Based on Bioscreen analysis, NO-np/GSH inhibited all growth for up to 8 hours as compared to NO-np, which completely inhibited growth for up to 4 hours. At 24 hours, there were significant decreases in percent survival for the NO-np as compared to control nanoparticles (np) (11.6% vs 68.6% survival; P value<0.0001) and NO-np/GSH as compared to rip with GSH (8.3% vs 77.3% survival; P value<0.0001) as determined by CFU (FIG. 2b). There was no statistically significant difference in cell survival at 24 hours between the NO-np treated and NO-np GSH treated (11.6% vs 8.3% survival; P value 0.18). GSH by itself was similar to control rip.

NO-np and NO-np with GSH Inhibit E. Coli Growth/Survival:

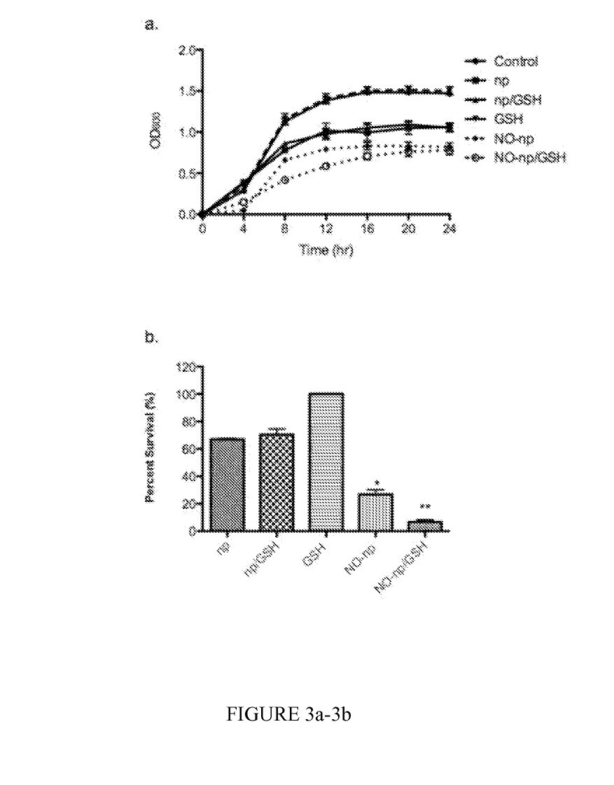

The effect of NO-np and NO-np/GSH on E. coli growth was determined in real-time for 24 h using Bioscreen C analysis (FIG. 3a). Both NO-np conditions significantly limited bacterial growth after 24 h co-incubation for all isolates, which correlated with CFU assays (FIG. 3b). Based on Bioscreen analysis, NO-np and NO-np/GSH inhibited growth similarly, though isolates treated with NO-np/GSH demonstrated a more gradual increase in OD. At 24 hours, there were significant decreases in percent survival for the NO-np as compared to the np (26.9% vs 67.0% survival; P value=0.003) and NO-np/GSH as compared to control np with GSH (6.8% vs 77.3% survival; P value=0.00001) as determined by CFU (FIG. 3b). There was a significant difference in cell survival between the NO-np and the NO-np GSH treated isolates (26.9% vs 6.8% survival; P value=0.0051), GSH alone was similar to PBS.

NO-np and NO-np with GSH Inhibit K. Pneumoniae Growth/Survival:

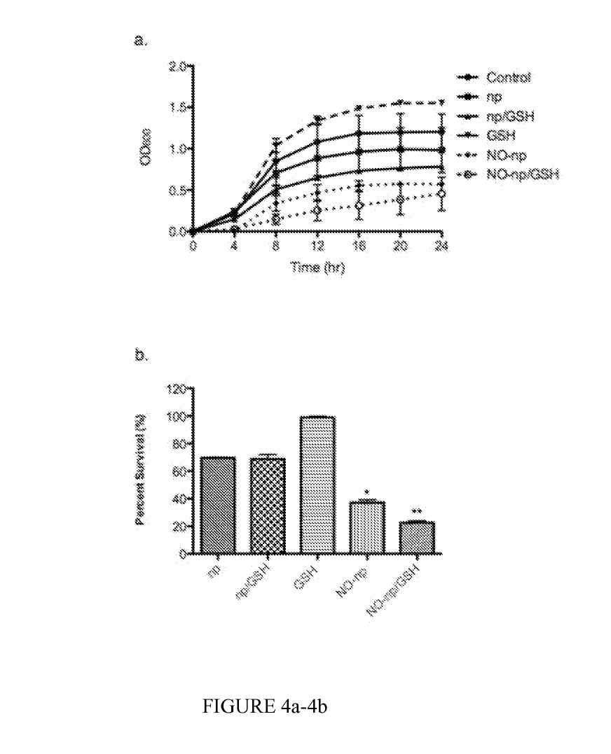

The effect of NO-np and NO-np/GSH on K. pneumoniae growth were determined in real-time for 24 h using Bioscreen C analysis (FIG. 4a). Both NO-np conditions completely inhibited growth for up to four hours, and both similarly and significantly limited bacterial growth after 24 h co-incubation for all isolates as compared to controls, Which correlated with CFU assays (FIG. 4b). After 24 hours, there were significant decreases in percent survival for the NO-np as compared to np (37.1% vs 69.5% survival; P value=0.0001) and NO-np/GSH as compared to np with GSH (22.5% vs 68.8% survival; P value=0.0002) as determined by CFU (FIG. 4b). There was a significant difference in cell survival between the NO-np and the NO-np/GSH treated isolates (37.1% vs 22.5% survival; P value<0.005). GSH alone was similar to PBS.

NO-np and NO-np with GSH Inhibit P. Aeruginosa Growth/Survival:

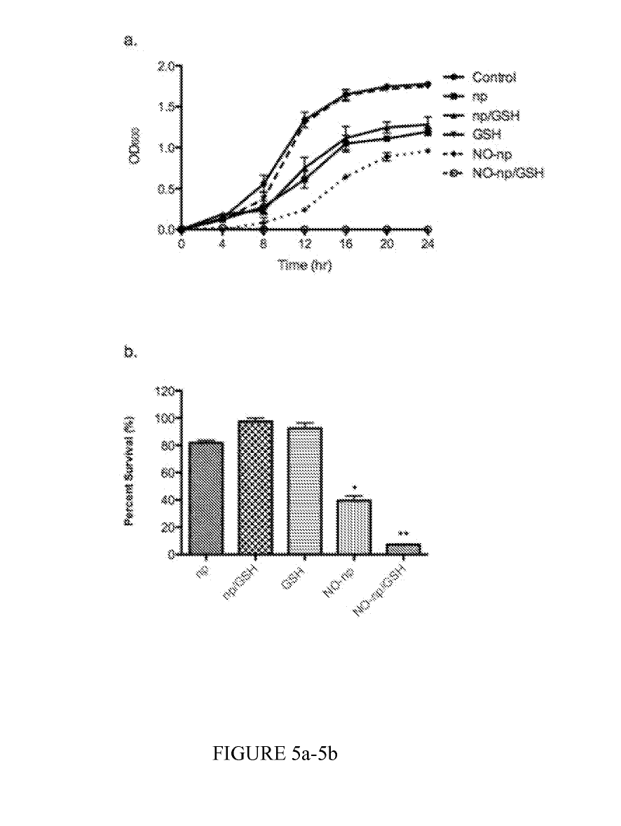

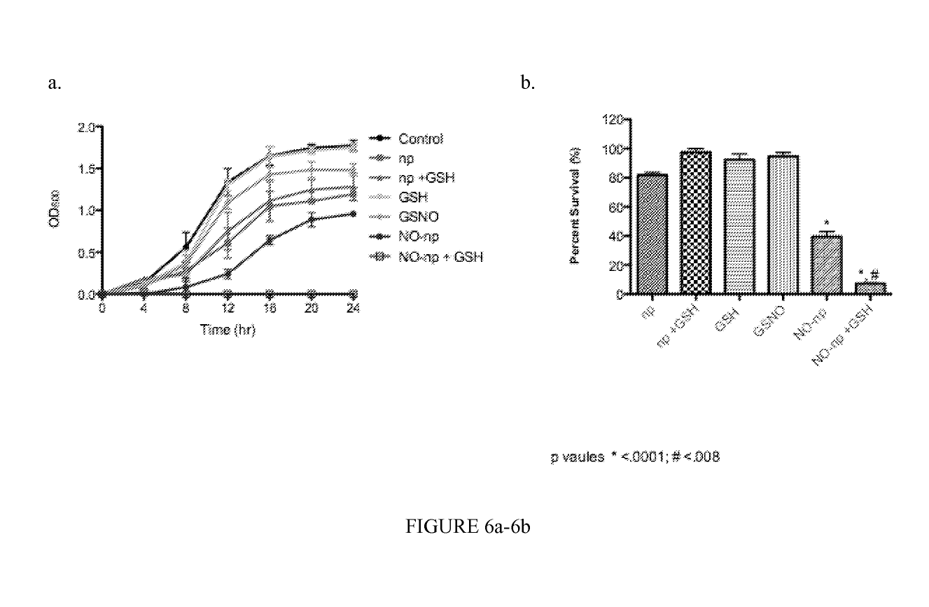

The effect of NO-np and NO-np/GSH on P. aeruginosa growth was determined in real-time for 24 h using Bioscreen C analysis (FIG. 5a). Both NO-np and NO-np/GSH completely inhibited growth for up to eight hours, however NO-np/GSH completely inhibited growth for 24 hours. After 24 hours, there were significant decreases in percent survival for the NO-np as compared to control np (39.5% vs 81.9% survival; P value=0.0001) and NO-np/GSH in comparison to np with GSH (7.2% vs 97.4% survival; P value<0.0001) as determined by CFU (FIG. 5b). The most significant difference in cell survival between the NO-np and the NO-np/GSH treated isolates was appreciated with these isolates (39.5% vs 7.2% survival; P value=0.0002). GSH was similar to PBS. See also FIG. 6.

Wound Healing Promotion by NO-np with GSH:

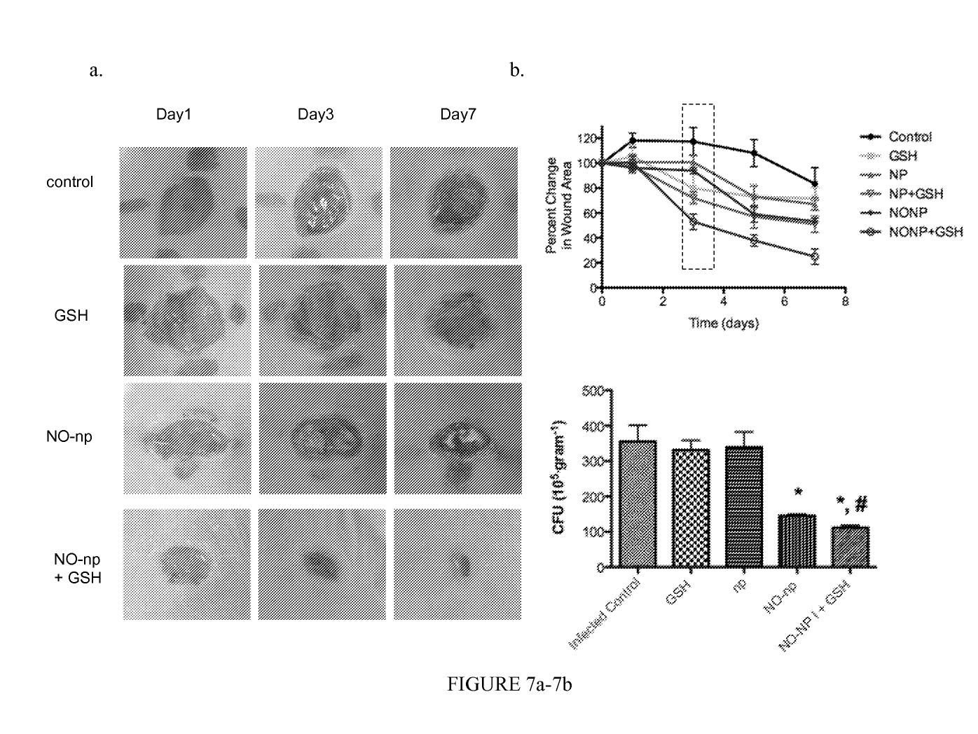

NO-np (10 mg/ml)+GSH (10 mM) is more effective than controls or NO-np alone in a Pseudomonal excisional wound mouse model (FIG. 7).

### Discussion

In light of the growing pathogen resistance to our armament of antibiotics, new directions in antimicrobial development must be pursued. The use of NO as an antimicrobial agent is elementary, as the means through which physiologic NO is generated and combats invading organisms is well understood [1; 2; 26]. Using NO-np, in vitro and in vivo bactericidal activity has been demonstrated against both Gram positive and negative organisms [20; 21; 22]. However, as NO is a versatile biomolecule in the living system, it is unclear to what extent its various intermediates and by-products are most effective in the various processes reliant on NO, such as host defense. To further elucidate this mechanism, in this study the role of NO-np generated GSNO was evaluated as an antimicrobial agent in vitro against various multi-drug resistant clinically relevant pathogens.

The present results show that when combined with GSH, NO-np are capable of forming GSNO and maintaining significant concentrations of GSNO over an extended period of time (>24 h). A mixture of nitrite and GSH also formed GSNO; however, the GSNO formed was relatively short-lived and decayed completely within six hours (data not shown). Therefore, the NO-np are an optimal platform to generate and maintain GSNO concentrations for durable periods of time, since the steady release of NO promotes the slow and steady formation of GSNO over an extended period of time.

GSNO's function as an antimicrobial agent has been previously reported [27]. In a study by Marcinkiwicz, a 5 mM concentration of GSNO was required to exert a MIC90 against E. coli (ATCC 25922). Based on HPLC data, the amount of GSNO generated when 5 mg/ml of NO-np are combined with 10 mM GSH is substantially less without sacrificing antimicrobial impact. The combined NO-np/GSH both significantly delayed/inhibited growth of all species investigated and/or limited survival as compared to controls and NO-np alone. P. aeruginosa isolates demonstrated the greatest sensitivity to GSNO, as there was no detectable growth over 24 hours based on Bioscreen C analysis and less than 10% cell survival by CFUs, whereas K. pneumoniae isolates were the most resistant. MRSA treated with both NO-np and NO-np/GSH demonstrated greater then MIC90 at 24 hours; however, there was a significant impact on growth kinetics between the two treatment groups, favoring NO-np/GSH. For all bacterial species tested, those treated with the NO-np/GSH exhibited significantly retarded growth curves even when compared to the NO-np treated, yet overall bacterial survival was less then 10%.

The impact of GSNO is likely two-fold. First, GSNO is well known as a NO-donor, and therefore may serve as a more stable reservoir of NO [13]. The activity of GSNO as an antimicrobial agent in this respect has been previously investigated and demonstrated [27]. Second, unlike NO, GSNO is a highly potent nitrosating agent, being able to transfer NO+ to an amine or thiol group to ultimately alter protein function.

In thinking of GSNO as an NO-donor, there is evidence suggesting that the degree of exposure to NO is important in determining bacterial cell survival. Moore et al. investigated the effects of NO on Bacillus subtilis and found that exposure to either 50 or 200 μM NO were tolerated by the bacteria with no significant loss in viability [28]. In contrast, lower concentrations of NO (20-25 μM) repetitively added over time led to a 100-fold reduction in Bacillus viability. These results imply that NO is a more effective antimicrobial when applied to bacteria over time as compared to a single bolus [28]. Continuous or repetitive exposure to NO may exhaust the actions of protective enzymes such as glutathione reductases or flavohemoglobins (hmp), and may explain the lack of resistance to NO-np, and even more so to NO-np/GSH, which provides a physiologic amount of NO in a controlled and sustained manner over 24 hours [25].

Interestingly, there were significant differences in survival noted between bacteria subjected to the NO-np and the NO-np/GSH. It is well established that enteric and uropathic pathogens have developed extensive scavenging mechanisms through which the harmful effects of NO can be dispelled, ranging from the above mentioned hmp to cytochrome c nitrite reductase to GSH-dependent formaldehyde dehydrogenase [23]. However, as demonstrated in Gram negative organisms such as Salmonella typhimurium, GSNO can be actively taken up and processed by microbial systems that typically function to import glutathione and other short peptides [29; 30; 31]. GSNO appears to be recognized as a substrate by the periplasmic enzyme glutamyltranspeptidase, which subsequently converts GSNO to S-nitrosocysteinyl-glycine. This nitrosated dipeptide in turn is imported into the bacterial cytoplasm across the inner membrane by a specialized dipeptide permease (Dpp). This Dpp is actually required for NO to exert a bactericidal impact.

In summary, it was demonstrated that GSNO can be effectively and efficiently generated from NO-np in the presence of GSH. The GSNO generated was shown to be an effective antimicrobial agent, even more so than NO alone, which is consistent with GSNO functioning as a potent nitrosating agent. The combination of NO-np with GSH presents a novel, facile, and effective means of generating GSNO to both allow for a better understanding of the physiologic and pathophysiologic mechanisms of NO. Moreover, the NO-np/GSH represents a potential broad-spectrum therapeutic that can impact multi-drug resistant pathogens.

### REFERENCES

[1] F. C. Fang, and A. Vazquez-Torres, Nitric oxide production by human macrophages: there's NO doubt about it. Am J Physiol Lung Cell Mol Physiol 282 (2002) L941-3.

[2] F. C. Fang, Perspectives series: host/pathogen interactions. Mechanisms of nitric oxide-related antimicrobial activity. J Clin Invest 99 (1997) 2818-25.

[3] M. W. Foster, D. T. Hess, and J. S. Stamler, Protein S-nitrosylation in health and disease: a current perspective. Trends in Molecular Medicine 15 (2009) 391-404.

[4] G. Han, J. H. Zippin, and A. Friedman, From Bench to Bedside: The Therapeutic Potential of Nitric Oxide in Dermatology. Journal of Drugs in Dermatology 8 (2009) 586-594.

[5] C. Potera, Nanoparticles Deliver Nitric Oxide Locally. Genetic Engineering & Biotechnology News 30 (2010) 12-+.

[6] T. Akaike, T. Okamoto, M. H. Zaki, S. Fujii, and T. Sawa, [New paradigm of host defense against intracellular pathogens by nitric oxide]. Nihon Hansenbyo Gakkai Zasshi 78 (2009) 41-7.

[7] A. Y. Coban, Antimicrobial effect of nitric oxide. Salud I Ciencia 16 (2008) 1340-1341.

[8] A. Vazquez-Torres, T. Stevanin, J. Jones-Carson, M. Castor, R. C. Read, and F. C. Fang, Analysis of nitric oxide-dependent antimicrobial actions in macrophages and mice. Methods Enzymol 437 (2008) 521-38.

[9] M. A. De Groote, and F. C. Fang, NO inhibitions: antimicrobial properties of nitric oxide. Clin Infect Dis 21 Suppl 2 (1995) S162-5.

[10] A. F. Tavares, L. S. Nobre, A. M. Melo, and L. M. Saraiva, A novel nitroreductase of Staphylococcus aureus with 5-nitrosoglutathione reductase activity. Journal of bacteriology 191 (2009) 3403-6.

[11] L. R. Jarboe, D. R. Hyduke, L. M. Tran, K. J. Y. Chou, and J. C. Liao, Determination of the Escherichia coli 5-nitrosoglutathione response network using integrated biochemical and systems analysis. Journal of Biological Chemistry 283 (2008) 5148-5157.

[12] A. M. Firoved, S. R. Wood, W. Ornatowski, V. Deretic, and G. S. Timmins, Microarray analysis and functional characterization of the nitrosative stress response in nonmucoid and mucoid Pseudomonas aeruginosa. Journal of bacteriology 186 (2004) 4046-50.

[13] A. R. Butler, F. W. Flitney, and D. L. Williams, NO, nitrosonium ions, nitroxide ions, nitrosothiols and iron-nitrosyls in biology: a chemist's perspective. Trends Pharmacol Sci 16 (1995) 18-22.

[14] L. M. Liu, A. Hausladen, M. Zeng, L. Que, J. Heitman, and J. S. Stamler, A metabolic enzyme for S-nitrosothiol conserved from bacteria to humans. Nature 410 (2001) 490-494.

[15] J. F. Logman, J. Stephens, B. Heeg, S. Haider, J. Cappelleri, D. Nathwani, A. Tice, and B. A. van Hout, Comparative effectiveness of antibiotics for the treatment of MRSA complicated skin and soft tissue infections. Curr Med Res Opin 26 (2010) 1565-78.

[16] C. A. Odell, Community-associated methicillin-resistant Staphylococcus aureus (CA-MRSA) skin infections. Curr Opin Pediatr 22 (2010) 273-7.

[17] M. S. Shenoy, G. K. Bhat, A. Kishore, and M. K. Hassan, Significance of MRSA strains in community associated skin and soft tissue infections. Indian J Med Microbiol 28 (2010) 152-4.

[18] R. Kock, K. Becker, B. Cookson, J. E. van Gemert-Pijnen, S. Harbarth, J. Kluytmans, M. Mielke, G. Peters, R. L. Skov, M. J. Struelens, E. Tacconelli, A. N. Tome, W. Witte, and A. W. Friedrich, Methicillin-resistant Staphylococcus aureus (MRSA): burden of disease and control challenges in Europe. Eurosurveillance 15 (2010) 12-20.

[19] H. Boucher, L. G. Miller, and R. R. Razonable, Serious infections caused by methicillin-resistant Staphylococcus aureus. Clinical Infectious Diseases 51 Suppl 2 (2010) 5183-97.

[20] M. R. Mihu, U. Sandkovsky, G. Han, J. M. Friedman, J. D. Nosanchuk, and L. R. Martinez, Nitrix oxide releasing nanoparticles are therapeutic for Acinetobacter baumanni wound infections. Virulence 1 (2010) 62-67.

[21] L. R. Martinez, G. Han, M. Chacko, M. R. Mihu, M. Jacobson, P. Gialanella, A. J. Friedman, J. D. Nosanchuk, and J. M. Friedman, Antimicrobial and Healing Efficacy of Sustained Release Nitric Oxide Nanoparticles Against Staphylococcus Aureus Skin Infection. Journal of Investigative Dermatology 129 (2009) 2463-2469.

[22] G. Han, L. R. Martinez, M. R. Mihu, A. J. Friedman, J. M. Friedman, and J. D. Nosanchuk, Nitric Oxide Releasing Nanoparticles Are Therapeutic for Staphylococcus aureus Abscesses in a Murine Model of Infection. Plos One 4 (2009).

[23] R. K. Poole, Nitric oxide and nitrosative stress tolerance in bacteria. Biochemical Society

Transactions 33 (2005) 176-180.

[24] G. Han, A. J. Friedman, and J. M. Friedman, Nitric oxide releasing nanoparticle synthesis and characterization. Methods Mol Biol 704 (2011) 187-95.

[25] A. J. Friedman, G. Han, M. S. Navati, M. Chacko, L. Gunther, A. Alfieri, and J. M. Friedman, Sustained release nitric oxide releasing nanoparticles: Characterization of a novel delivery platform based on nitrite containing hydrogel/glass composites. Nitric Oxide-Biology and Chemistry 19 (2008) 12-20.

[26] A. R. Richardson, S. J. Libby, and F. C. Fang, A nitric oxide-inducible lactate dehydrogenase enables Staphylococcus aureus to resist innate immunity. Science 319 (2008) 1672-6.

[27] J. Marcinkiewicz, Nitric oxide and antimicrobial activity of reactive oxygen intermediates. Immunopharmacology 37 (1997) 35-41.

[28] C. M. Moore, M. M. Nakano, T. Wang, R. W. Ye, and J. D. Helmann, Response of Bacillus subtilis to Nitric Oxide and the Nitrosating Agent Sodium Nitroprusside. J. Bacteriol. 186 (2004) 4655-4664.

[29] M. A. De Groote, U. A. Ochsner, M. U. Shiloh, C. Nathan, J. M. McCord, M. C. Dinauer, S. J. Libby, A. Vazquez-Torres, Y. Xu, and F. C. Fang, Periplasmic superoxide dismutase protects Salmonella from products of phagocyte NADPH-oxidase and nitric oxide synthase. Proc Natl Acad Sci USA 94 (1997) 13997-4001.

[30] M. A. De Groote, T. Testerman, Y. Xu, G. Stauffer, and F. C. Fang, Homocysteine antagonism of nitric oxide-related cytostasis in Salmonella typhimurium. Science 272 (1996) 414-7.

[31] M. A. De Groote, D. Granger, Y. Xu, G. Campbell, R. Prince, and F. C. Fang, Genetic and redox determinants of nitric oxide cytotoxicity in a Salmonella typhimurium model. Proc Natl Acad Sci USA 92 (1995) 6399-403.

What is claimed is:

1. A method for enhancing the efficacy of nitric oxide (NO) released from NO releasing nanoparticles (NO-np) comprising combining the NO-np with exogenous glutathione (GSH) so as to enhance the efficacy of NO that is released.

2. The method of claim 1, wherein S-nitrosoglutathione (GSNO) is formed by reaction of NO with GSH.

3. The method of claim 1, wherein GSH is dissolved in a carrier mixed with NO-np.

4. The method of claim 1, wherein GSH is encapsulated in nanoparticles (GSH-np) and mixed with NO-np.

5. A method of treating a microbial infection comprising applying NO to the microbes by combining NO-np and GSH according to the method of claim 1.

6. The method of claim 5 Wherein the infection is a bacterial, viral, fungal or parasitic infection.

7. The method of claim 6, wherein the bacteria are methicillin resistant Staphylococcus aureus (MRSA), Escherichia coli, Klebsiella pneumoniae, or Pseudomonas aeruginosa.

8. A method of promoting wound healing or hair growth, or treating a burn in a subject comprising applying NO to the wound, hair or burn by combining NO-np and GSH according to the method of claim 1.

9. A method of promoting angiogenesis or vasodilation in a subject comprising administering NO to the subject by combining NO-np and GSH according to the method of claim 1.

10. A method of treating erectile dysfunction in a subject comprising topically applying NO to the penis of the subject by combining NO-np and GSH according to the method of claim 1.

11. A method of administering nitric oxide (NO) to a subject comprising administering to the subject a combination of NO releasing nanoparticles (NO-np) and exogenous glutathione according to the method of claim 1.

12. The method of claim 11, wherein the subject has a cardiovascular disorder, a pulmonary disorder, peripheral vascular disease, erectile dysfunction, scleroderma, sickle cell anemia, a microbial infection, a wound, a burn, an inflammatory skin disease, psoriasis or eczema.

13. A composition for delivering nitric oxide (NO) comprising NO releasing nanoparticles (NO-np) and glutathione (GSH).

14. The composition of claim 13, wherein S-nitrosoglutathione (GSNO) is formed by reaction of NO with glutathione.

15. The composition of claim 13, wherein GSH is dissolved in a carrier mixed with NO-np.

16. The composition of claim 13, GSH is encapsulated in nanoparticles (GSH-np) and mixed with NO-np.

17. An antimicrobial agent, a wound healing accelerant, a pro-erectile agent, an anti-hypertensive agent, a pro-resuscitive agent, a blood storage stabilizer, an anti-vasospasmic agent, a chemotherapeutic agent, an immunomodulatory agent, or an anti-aging agent comprising the composition of claim 13.
